(19) United States
(12) Plant Patent Application Publication
(10) Pub. No.: US 2026/0157248 P1
(43) Pub. Date: Jun. 4, 2026

(54) Aechmea plant named 'A2191'

(50) ;

(71) Applicant: Chester Skotak, Alajuela (CR)

(72) Inventor: Chester Skotak, Alajuela (CR)

(21) Appl. No.: 19/401,690

(22) Filed: Nov. 26, 2025

(30) Foreign Application Priority Data
Nov. 29, 2024 (QZ) 2024/2861

(51) Int. Cl. A01H 6/22; A01H 5/02; A01H 5/12

(52) U.S. Cl. CPC A01H 6/22; A01H 5/02; A01H 5/12
USPC PLT/370

(57) ABSTRACT

A new and distinct Aechmea plant named ‘A2191’ particularly distinguished by a highly intense, deep red inflorescence coloration; a compact growth habit with small and dense rosettes; a unique leaf pattern characterized by strong and regular silver banding on dark green foliage; a uniform and vigorous plantlet development under tissue culture; and a high ornamental value due to its contrasting leaf colorations and proportional plant form.

Genus and species:

Aechmea chantinii.

Variety denomination:

‘A2191’.

### STATEMENT REGARDING PRIOR DISCLOSURES BY THE INVENTOR

This application claims priority to CPVO application no. 2024/2861 filed on Nov. 29, 2024, which is herein incorporated by reference. The Applicant asserts that no publications or advertisements relating to sales, offers for sale, or public distribution occurred more than one year prior to the effective filing date of this application. Any information about the claimed plant would have been obtained from a direct or indirect disclosure from the Inventor. The Applicant claims a prior art exemption under 35 U.S.C. 102(b)(1) for disclosures and/or sales that fall within a one-year grace period prior to the effective filing date.

BACKGROUND OF THE NEW PLANT

The present invention comprises a new and distinct variety of Aechmea plant, botanically known as Aechmea chantinii, and hereinafter referred to by the variety name ‘A2191’.

Aechmea comprises a genus of more than 168 species of evergeen perennials suitable for cultivation in the home or in the greenhouse. Aechmea may be terrestrial or epiphytic. For the most part, the species vary in diameter from 12 to 18 inches to 3 or 4 feet and have rosettes of spiny-edged leaves.

Flowers and bracts of Aechmea frequently have brilliant colors and may last for several months. The range of colors for Aechmea is generally from the yellow through orange but may also include pink, orange, red and red-purple. Tubular, three-petalled flowers may also appear but are usually short-lived.

Aechmea may be grown as pot plants for greenhouse or home use. Typically, the plants are shaded from direct sunlight; and the central, vaselike part of the leaf rosette is normally filled with water.

Aechmea is native to tropical America. Leaves of Aechmea are usually formed from basal rosettes which are entire and in several vertical ranks. Aechmea has terminal spikes or panicles which are often bracted with petals united in a tube longer than the calyx.

Asexual propagation of Aechmea is frequently done through meristematic tissue culture. Propagation can also be from offshoots which can be detached from the mother plant and grown in an appropriate soil or bark mixture. Methods for cultivating and crossing of Aechmea are well known.

The new Aechmea variety ‘A2191’ is the product of a controlled cross pollination conducted by the inventor in Alajuela, Costa Rica in May 2014. The objective of the breeding program was to develop compact and uniform Aechmea chantinii plants with unique silver variegation on the leaves, deep red inflorescence, and plants suitable for pot production. The female or seed parent was an unnamed and unpatented Aechmea chantinii breeding line. The male or pollen parent was an unnamed and unpatented Aechmea chantinii breeding line. ‘A2191’ was selected by the inventor in June 2018 in Alajuela, Costa Rica as a single flowering plant from within the progeny of the stated cross.

The new Aechmea cultivar “A2191’ was first reproduced asexually by meristematic tissue culture in March 2020 at a commercial laboratory in Evergem, Belgium. Asexual propagation by meristematic tissue culture of the new variety has shown that the unique features of the new variety are stable and reproduced true-to-type to date through 5 successive generations.

### SUMMARY OF THE INVENTION

The new Aechmea cultivar ‘A2191’ has not been observed under all possible environmental conditions. The phenotype may vary somewhat with variations in environment such as temperature, day length, light intensity, water status, fertilizer rate and type, without, however, any variance in genotype.

The following are the most outstanding and distinguishing characteristics of this new Aechmea cultivar. The combination of these characteristics distinguishes ‘A2191’ as a new and distinct variety of Aechmea.
1. Aechmea ‘A2191’ exhibits highly intense, deep red inflorescence coloration.
2. Aechmea ‘A2191’ exhibits a compact growth habit with small and dense rosettes.
3. Aechmea ‘A2191’ exhibits a unique leaf pattern characterized by strong and regular silver banding on dark green foliage.
4. Aechmea ‘A2191’ exhibits a uniform and vigorous plantlet development under tissue culture.
5. Aechmea ‘A2191’ exhibits high ornamental value due to its contrasting leaf colorations and proportional plant form.

### DESCRIPTION OF THE PHOTOGRAPHS

This new Aechmea cultivar is illustrated by the accompanying-colored photographs which show the overall appearance and distinct characteristics of the plant. The colors shown are as true as can be reasonably obtained by conventional photographic procedures. The photographs are of 12-month-old plants grown in 12-centimeter circular containers in a conventional glass greenhouse in Evergem, Belgium. Colors in the photographs may differ slightly from the color values cited in the botanical description which accurately describes the colors of the new variety.

FIG. 1 shows the overall plant form, foliage, and inflorescence of ‘A2191’.

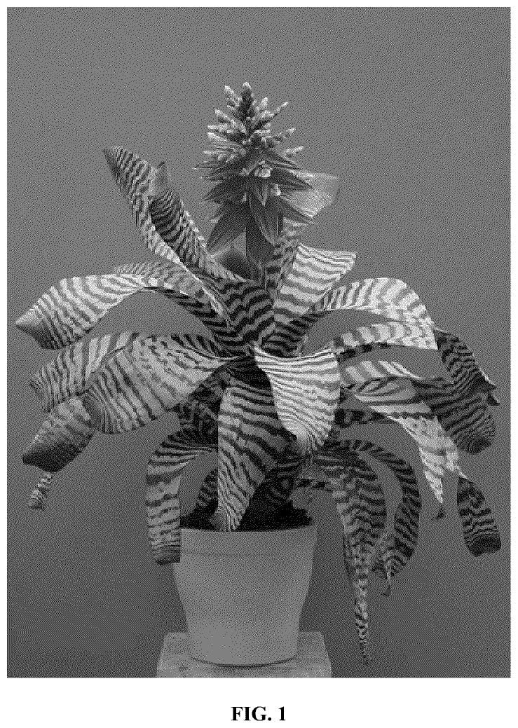

FIG. 2 shows a close-up of a typical leaf of ‘A2191’.

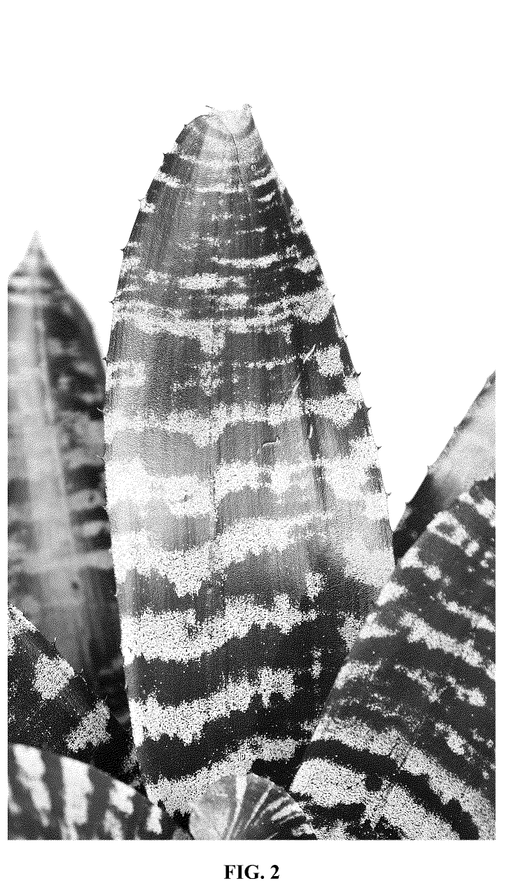

FIG. 3 shows a close-up of typical inflorescence of ‘A2191’.

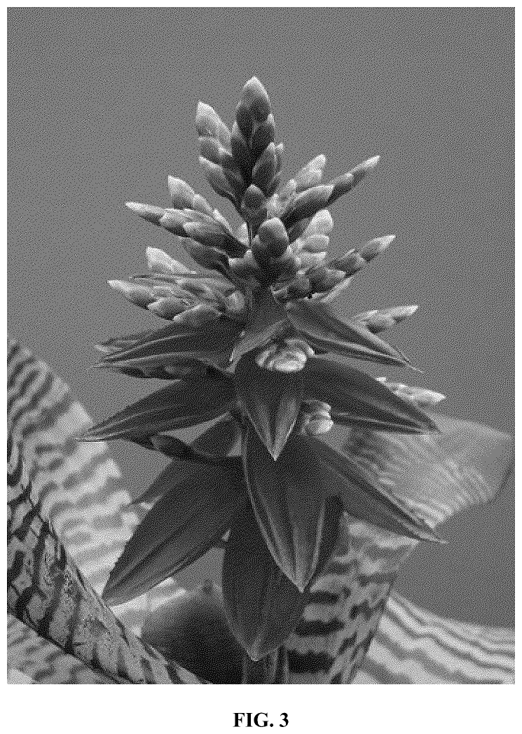

### DESCRIPTION OF THE NEW VARIETY

In the following description, color references are made to The Royal Horticultural Society Colour Chart, Sixth Edition, except where general color terms of ordinary dictionary significance are used.

The following observations and measurements describe 12-month-old plants grown in 12-centimeter circular containers in a conventional glass greenhouse located in Evergem, Belgium under normal light levels with no supplemental lighting. Average daily temperatures ranged between 21° C. and 23° C. and average nightly temperatures ranged between 19° C. and 21° C. Measurements and numerical values represent averages of typical plant types.

### DETAILED BOTANICAL DESCRIPTION

Classification:
   Family.—Bromeliaceae.
   Botanical.—Aechmea chantinii
   Denomination.—‘A2191’.
General description:
   Plant type.—Tropical perennial suitable as an indoor potted plant.
   Growth habit.—Highly symmetrical, compact and dense basal rosette; inflorescence grows from the center of the basal rosette.
   Overall plant shape.—Obovate with a flattened top, inflorescence carried slightly above the foliar plane.
   Height from soil level to top of foliar plane.—27.2 cm.
   Plant diameter or area of spread.—27.7 cm.
   Growth rate.—Moderate.
   Plant vigor.—Moderate.
   Branching habit.—No lateral branching; leaves in a basal rosette, inflorescence grows from the center of the basal rosette.
   Propagation type.—Meristematic tissue culture.
   Time to produce a finished plant.—Approximately 52 weeks from a tissue cultured plantlet.
Root description:
   General.—Slightly fleshy and very slightly fibrous.
   Length.—Average 6.8 cm.
   Width.—Average 0.1 cm.
   Color.—166C and 166D (greyed-orange).
Foliage description:
   Arrangement.—Rosulate.
   Compound or single.—Single.
   Quantity of leaves per rosette.—23.
   Lamina.—Shape: Oblong. Length: Average 22.6 cm. Width: Average 4.9 cm. Aspect: Flat to slightly concave towards the base, slightly twisted axially with older leaves curled slightly downwards. Apex shape: Broad and abruptly acute with a mucronulate tip. Base: Sheathing. Sheath length: Average 5.0 cm. Sheath width Average 5.9 cm. Sheath color: N148C (yellow-green) and edged 144A (yellow-green). Margin: Dentate with sharp teeth. Undulation: Coarsely undulate and slightly twisted axially. Lobes: Unlobed. Texture (both upper and lower surfaces): Smooth and glabrous. Rugosity: Non-rugose. Pubescence: No pubescence present. Luster: Upper surface: Glossy. Lower surface: Slightly glossy. Color: Juvenile foliage: Upper surface: A blend of NN137A and 147A (green to yellow-green) and blotched N155A and 198C (white to green-white) in radial lines across the lamina. Lower surface: A blend of 137B and 146A (green to yellow-green) and heavily blotched N155A and 198D (white to green-white) in radial lines across the lamina. Mature foliage: Upper surface: Slightly darker than a blend of NN137A and 147A (green to yellow-green), closest to 147A, and blotched N155A (white) in loosely arranged radial lines across the lamina. Lower surface: NN137B (green) and heavily blotched N155A (white) in loosely arranged radial lines across the lamina. Venation: Pattern: Parallel. Color (mature foliage): Upper surface: Upper surface venation is colored the same as the upper surface of the lamina. Lower surface: Lower surface venation is colored the same as the lower surface of the lamina.
Inflorescence description:
   Inflorescence position and height.—Upright and above foliage.
   Inflorescence size.—Compact and proportionate.
   Inflorescence color.—46A to 47A (red).
   Intensity of inflorescence color.—High.
   Floral bract width.—Broad.
   Bract color.—Deep red.
   Flower density.—High.
Fruit and seed set: None observed to date.
Temperature tolerances: USDA Zones 10 to 13; at least tolerant of temperatures to 40 degrees Celsius.
Rain tolerance: High.
Wind tolerance: High.
Disease and pest tolerance: No claims are made to any superior disease or insect resistance or tolerance.

### COMPARISON WITH THE PARENT PLANTS

The new Aechmea cultivar ‘A2191’ can be distinguished from its female or seed parent in the following characteristics described in Table 1 below.

TABLE 1

| Characteristic | ‘A2191’ | Female parent |
|---|---|---|
| Overall plant | Compact and | Medium and |
| habit | upright rosette | open rosette |
| Leaf surface | Strong and uniform | Weak silver |
| pattern | silver banding | banding |
| Leaf color | Dark green with | Green, slightly |
| (upper surface) | silver bands | dull |
| Inflorescence | Upright and above | Partly within |
| position | foliage | rosette |
| Inflorescence | 46A to 47A | Orange-red |
| color | (deep red) | |
| Floral bract | Broad | Narrow |
| width | | |
| Overall | Compact and | Moderately |
| appearance | uniform | vigorous |

The new Aechmea cultivar ‘A2191’ can be distinguished from its male or pollen parent in the following characteristics described in Table 2 below.

TABLE 2

| Characteristic | ‘A2191’ | Male Parent |
|---|---|---|
| Overall plant | Compact and | More spreading |
| habit | upright rosette | rosette |
| Leaf banding | Strong and uniform | Irregular and |
| | silver banding | weak |
| Leaf color | Dark green with | Light green |
| (upper surface) | silver overlay | |
| Inflorescence | Upright and above | Medium |
| position | foliage | |
| Inflorescence | 46A to 47A | Orange-red |
| color | (deep red) | |
| Flower density | High | Moderate |
| Plant vigor | Strong and uniform | Moderate |

### COMPARISON WITH RELATED KNOWN VARIETY

The new Aechmea cultivar ‘A2191’ can be distinguished from the related known variety of Aechmea ‘Pepita’ in the following characteristics described in Table 3 below.

TABLE 3

| Characteristic | ‘A2191’ | ‘Pepita’ |
|---|---|---|
| Overall plant | Compact and | Larger, more spreading |
| habit | upright rosette | rosette |
| Leaf banding | Strong and uniform | Absent or weak |
| | silver banding | |
| Leaf color | Dark green with | Green |
| (upper surface) | silver overlay | |
| Inflorescence | Compact and | Medium |
| size | proportionate | |
| Inflorescence | 46A to 47A | Orange-red |
| color | (deep red) | |
| Bract color | Deep red | Light-red |
| Overall | Highly uniform and | Moderate uniformity |
| appearance | ornamental | |

What is claimed is:

1. A new and distinct cultivar of Aechmea plant named ‘A2191’, substantially as illustrated and described herein.
